(19) United States
(12) Patent Application Publication
(10) Pub. No.: US 2009/0137686 A1
(43) Pub. Date: May 28, 2009

(54) Treatment of Ophthalmic Diseases

(76) Inventors: Alan Rothaul, Cambridge (GB); John Brew, Essex (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/094,505

(22) PCT Filed: Nov. 24, 2006
(86) PCT No.: PCT/GB06/04410

§ 371 Date: Aug. 25, 2008

(30) Foreign Application Priority Data
Nov. 24, 2005 (GB) 0523964.5

(51) Int. Cl. A61K 31/138; A61P 27/00

(52) U.S. Cl. 514/653

(57) ABSTRACT

Beta-amino alcohols are useful for the treatment of ophthalmic conditions.

### FIELD OF THE INVENTION

This invention relates to the use of beta-amino alcohols and isomers for the treatment of ophthalmic diseases characterized by ocular inflammation, dry eye disorders, increased intra-ocular pressure, pathologic ocular angiogenesis and/or retina or sub-retinal edema.

### BACKGROUND OF THE INVENTION

Diseases and degenerative conditions of the optic nerve and retina are the leading causes of blindness in the world. A significant degenerative condition of the retina is age-related macular degeneration (ARMD). ARMD is the most common cause of blindness in people over 50 in the USA and its prevalence increases with age. ARMD is classified as either wet (neovascular) or dry (non-neovascular); the dry form of the disease is more common. Macular degeneration occurs when the central retina has become distorted and thinned usually associated with age but also characterised by intra-ocular inflammation and angiogenesis (wet ARMD only) and/or intra-ocular infection.

Retinopathy associated with diabetes is a leading cause of blindness in type 1 diabetes, and is also common in type II diabetes. The degree of retinopathy depends on the duration of diabetes, and generally begins to occur ten or more years after onset of diabetes. Diabetic retinopathy may be classified as non-proliferative, where the retinopathy is characterised by increased capillary permeability, edema and exudates, or proliferative, where the retinopathy is characterised by neovascularisation extending from the retina to the vitreous, scarring, deposit of fibrous tissue and the potential for retinal detachment. Diabetic retinopathy is believed to be caused by the development of glycosylated proteins due to high blood glucose. The subsequent generation of free radicals, resulting in oxidative tissue damage, local inflammation and production of growth factors (such as VEGF and FGF) and inflammatory mediators, leads to inappropriate neovascularisation in common with the wet form of ARMD. Several other less common retinopathies include choroidal neovascular membrane (CNVM), cystoid macular edema (CME), epi-retinal membrane (ERM) and macular hole.

Currently, no drugs are approved for the treatment of diabetic retinopathy or macular edema. The current standard treatment is laser photocoagulation which, by destroying local tissue, decreases the production of cytokines and growth factors but is unfortunately cytodestructive and causes permanent impairment of vision. These neovasular diseases have the potential to be treated with angiostatic agents in combination with anti-inflammatory drugs.

Dry eye, or keratoconjunctivitis, is a common opthalmological disease affecting millions of Americans each year. The condition is particularly prevalent in post-menopausal women due to hormonal changes caused by the cessation of fertility. Dry eye is primarily caused by the break-down of the pre-ocular tear film which results in dehydration of the exposed outer surface. There is a strong rationale that ocular inflammation as a result of pro-inflammatory cytokines and growth factors plays a major role in the underlying causes of dry eye. As such, locally administered anti-cytokine or general anti-inflammatory agents are often used in the treatment of dry eye.

Another disease of the interior of the eye is uveitis, or inflammation of the uveal tract. The uveal tract (uvea) is composed of the iris, ciliary body and choroid. Uveitis may be caused by trauma, infection or surgery and can affect any age group. The disease is classified anatomically as anterior, intermediate, posterior or diffuse. Anterior uveitis affects the anterior portion of the eye including the iris. Intermediate uveitis, also called peripheral uveitis, is centred in the area immediately behind the iris and lens in the region of the ciliary body. Posterior uveitis may also constitute a form of retinitis, or it may affect the choroids and the optic nerve. Diffuse uveitis involves all parts of the eye. The most common treatment of uveitis is with locally administered glucocorticosteroids, often in combination with other anti-inflammatory drugs. Although these drugs are effective in the treatment of many forms of ocular inflammation, they have several side-effects including endophthalmitis, cataracts and elevated intra-ocular pressure (IOP). There is a need for potent anti-inflammatory agents with an improved side-effect profile, the so-called non-steroid steroid, for the treatment of ophthalmic inflammation and edema.

Glaucoma is made up of a collection of eye diseases that cause vision loss by damage to the optic nerve. Elevated intraocular pressure (IOP) due to inadequate ocular drainage is the primary cause of glaucoma. Glaucoma often develops as the eye ages, or it can occur as the result of an eye injury, inflammation, tumour or in advanced cases of cataract or diabetes. As discussed above it can also be caused by the increase in IOP caused by treatment with steroids. Drug therapies that are proven to be effective in glaucoma reduce IOP either by decreasing vitreous humor production or by facilitating ocular draining. Such agents are often vasodilators and as such act on the sympathetic nervous system and include adrenergic antagonists. There is a need for agents that can be delivered locally to the eye with minimal systemic side-effects.

Beta-amino alcohols include compounds of general formula (1):

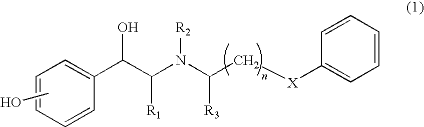

wherein R₁ is H or Me; R₂ is H or alkyl and can be part of a ring with R₃; R₃ is H, Me or CH₂ (when forming part of a ring with R₂); n=0-2; X is CH₂ or O; and the two phenyl groups may be optionally substituted with OH, OMe, halogen, NHCHO, NHSO₂Me, CONH₂ or SOMe.

WO2005/089741 discloses the use of compounds of general formula (1) for the treatment of general inflammatory conditions and pain.

### SUMMARY OF THE INVENTION

This invention is the use of compounds of general formula (1) for the treatment of ophthalmic conditions, including any of those described above.

A large number of compounds of formula (1) suffer from extensive first-pass metabolism which makes the compounds ideal for topical delivery where systemic side-effects can be minimised. The unexpected profile of these beta amino alcohols and their isomers is consistent with the non-steroid steroid and provides cytokine modulation with down-regulation of pro-inflammatory cytokines (such as TNFα and IL-1β) and growth factors involved in neovascularisation (such as FGF and VEGF) and upregulation of anti-inflammatory cytokines (such as IL-10), sometimes in combination with adrenoreceptor blocking activity resulting in vasodilation.

### DESCRIPTION OF THE INVENTION

In this specification, reference to “treatment” includes any form of therapy, including curative and prophylactic. Reference to compounds of formula (1) includes the free base form, salts, e.g. the hydrochloride, metabolites and pro-drugs thereof, as well as any diastereomers and enantiomers, and racemic or non-racemic mixtures. An alkyl group or ring typically contains up to 6 atoms.

Local administration of a beta amino alcohol (1), or an isomer or a metabolite thereof, allows the treatment of a range of ophthalmic diseases such as ARMD, retinopathies (including diabetic retinopathy), dry eye, uveitis and even glaucoma with minimal systemic side-effects.

Some isomers of formula (1) have vasodilator properties, by antagonism at adrenoceptors. These agents may be particularly interesting in the treatment of glaucoma but less preferred for the treatment of ocular inflammatory diseases.

A preferred diastereomer or enantiomer or non-racemic mixture, e.g. for the treatment of ARMD, retinopathies (including diabetic retinopathy), dry eye and uveitis has cytokine/growth factor modulatory activity but little or no activity at the adrenoreceptors. This activity may be determined by use of the appropriate in vitro and in vivo assays.

Compounds of formula (1) may be used according to the invention when the patient is also being administered, or in combination with, another therapeutic agent selected from angiostatic peptides, such as angiostatin; angiostatic steroids, such as anecortave acetate; modulators of VEGF or FGF, such as zactima; non-steroidal anti-inflammatory drugs (NSAIDs) formulated for ocular use such as flurbiprofen, diclofenac and ketorolac; glucocorticosteroids such as methylprednisolone; leukotriene modulators such as zilueton; anti-histamines such as cetirizine, loratidine and ketotifen; and general cytokine/growth factor-modulating agents such as cyclosporin A, diacerein, tetracyclines, fluoroquinolone antibiotics, quinoline antimalarials, statins and phosphodiesterase inhibitors. Compounds of formula (1) may be administered before, during or after laser photocoagulation therapy.

The preferred route of administration is topical to the eye. Another route of application is intraocular injection.

Formulations of compounds (1) suitable for topical application to the eye or intraocular injection are known to those of ordinary skill in the art. The dose of the active agent will depend on the nature and degree of the condition, the age and condition of the patient and other factors known to those skilled in the art. A typical dose is 0.01-100 mg given one to three times per day.

The following experimental work illustrates the invention. This involves an in vitro whole blood assay stimulated with Staphylococcus epidermidis, measuring IFNγ. IFNγ as a key mediator in pathological processes in ocular inflammatory disease, namely age-related macular degeneration (Penfold et al, 2002) and proliferative vitreoretinopathy (El-Ghrably et al, 2001).

1:100 diluted heat-killed Staphylococcus epidermidis in conjunction with various concentrations of test compound (in a stock DMSO solution) was added to a 100-fold volume of whole blood. After 24 hours, whole blood was diluted 1:4 and IFNγ was measured by enzyme-linked immuno-absorptive assay.

It was found that ritodrine racemate and ifenprodil erythro racemate both have strong inhibitory activity of IFNg secretion resulting from Staphylococcus epidermidis stimulation. Both compounds had a strong inhibitory effect at their lowest concentration of 62.5 nMol, with IFNγ concentrations cut from 4500 pg/ml to 1500 pg/ml; 66% inhibition.

1. A method for treating an ophthalmic condition, wherein said method comprises administering, to a patient in need of such treatment, a compound of formula 1 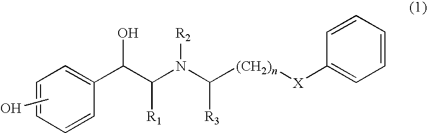
wherein R₁ is H or Me;
R₂ is H or alkyl or is part of a ring with R₃;
R₃ is H, Me or CH₂ (when forming part of a ring with R₂);
n=0-2;
X is CH₂ or O; and
either or each of the two phenyl groups is optionally substituted with OH, OMe, halogen, NHCHO, NHSO₂Me, CONH₂ or SOMe;
or a salt thereof.

2. The method according to claim 1, wherein the ophthalmic condition is age-related macular degeneration.

3. The method according to claim 1, wherein the ophthalmic condition is diabetic retinopathy.

4. The method according to claim 1, wherein the ophthalmic condition is choroidal neovascular membrane, cystoid macular edema, epi-retinal membrane or macular hole.

5. The method according to claim 1, wherein the ophthalmic condition is dry eye.

6. The method according to claim 1, wherein the ophthalmic condition is uveitis.

7. The method according to claim 1, wherein the compound is in the form of an enantiomer or diastereomer that has little or no activity at adrenoceptors.

8. The method according to claim 1, wherein the ophthalmic condition is glaucoma.

9. The method according to claim 8, wherein the compound is in the form of an enantiomer or diastereomer that also has adrenoceptor antagonist activity.

10. The method according to claim 8, wherein the compound is administered as a non-racemic mixture where the majority of the mixture is an enantiomer or diastereomer that has little or no activity at adrenoceptors.

11. The method according to claim 1, wherein the compound is administered before, during or after laser photocoagulation therapy.

12. The method according to claim 1, wherein the compound is administered to the eye topically or by intraocular injection.

13. The method according to claim 1, wherein the patient to be treated is also administered another therapeutic agent selected from angiostatic peptides, angiostatic steroids, modulators of VEGF or FGF, non-steroidal anti-inflammatory drugs formulated for ocular use, glucocorticosteroids, leukotriene modulators, antihistamines and general cytokine/growth factor-modulating agents.

14. The method according to claim 13, wherein compound (1) and said another agent are provided in combination.
